(19) United States
(12) Patent Application Publication
(10) Pub. No.: US 2021/0251580 A1
(43) Pub. Date: Aug. 19, 2021

(54) BLOOD PRESSURE AND BLOOD GLUCOSE MEASUREMENT AND TRACKING SYSTEM AND METHOD

(71) Applicant: mmHg Inc., Edmonton (CA)

(72) Inventors: Raj PADWAL, Edmonton (CA); William MOTT, Edmonton (CA); Liam HODGINS, Edmonton (CA); Peter WOOD, Edmonton (CA); Jennifer RINGROSE, Edmonton (CA)

(21) Appl. No.: 17/175,333

(22) Filed: Feb. 12, 2021

Related U.S. Application Data
(60) Provisional application No. 62/976,130, filed on Feb. 13, 2020.

(51) Int. Cl. A61B 5/00; A61B 5/021; A61B 5/024; A61B 5/145; A61B 5/01; A61B 5/08

(52) U.S. Cl. CPC A61B 5/7475; A61B 5/743; A61B 5/021; A61B 5/08; A61B 5/14532; A61B 5/01; A61B 5/024

(57) ABSTRACT

A computing device with a touch sensitive display is used to graphically display point-of-care diagnostic health data values (e.g., blood pressure or blood glucose readings) in a calendar view. The user performs gestures on the touch sensitive display to interact with the calendar to select one or more series of the data from specific calendar days. Computations are performed on the selected series to summarize the data. Data from one or multiple different series are organized, summarized and displayed in a graphical, numerical, or tabular format to enable comparisons between series, and facilitate understanding of data and data trends for diagnostic and treatment decisions.

### CROSS-REFERENCE TO RELATED APPLICATION

This application claims priority to U.S. provisional application Ser. No. 62/976,130, filed on Feb. 13, 2020, the entire contents of which are hereby incorporated by reference herein.

### FIELD OF THE INVENTION

The present invention relates generally to computer-implemented methods and systems to acquire, analyze, and display one or more blood pressure and/or blood glucose series from stored blood pressure or blood glucose data.

### BACKGROUND

High blood pressure (hypertension) is a leading cause of death and disability and a major risk factor for cardiovascular disease, kidney disease, and dementia. Although a patient's blood pressure has traditionally been measured in a clinic setting for diagnostic or management purposes, increasing emphasis is being placed on out-of-office measurement, most commonly home blood pressure measurement. Home blood pressure readings are more prognostically accurate than clinic readings, eliminate biases that occur in the clinic setting with blood pressure being spuriously high (‘white coat hypertension or white coat effect’) or low (‘masked hypertension or masked effect’). Home blood pressure monitoring encourages self-management and medication adherence.

High blood glucose (diabetes) is a major risk factor for cardiorenal disease, neuropathy, and blindness. High blood glucose levels are associated with poorer outcomes; therefore, blood glucose monitoring is important for assessing risk and guiding therapy. Blood glucose monitoring is most commonly done in intermittent fashion by performing serial chemstrip tests. Patients are often required to test before meals or after meals in order to obtain a comprehensive assessment of their circadian glucose trends. Although patients usually record this in written form or electronic form, it is often difficult to quickly calculate means and appreciate trends in the data. Such insights can be crucial to determining the next course of action in terms of clinical management.

Outside of the clinical arena, consumers are becoming more interested in monitoring health biometrics such as blood pressure and blood glucose to track their health status and wellbeing. Therefore, blood pressure and blood glucose monitoring have become commonly performed biometric tracking activities. In addition, other biometrics such as oxygen saturation, weight, body temperature, and heart rate, can all be used to support blood pressure or blood glucose data for diagnostic and prognostic purposes.

Because blood pressure and blood glucose exhibit a high degree of variability, constantly changing in response to a variety of endogenous and exogenous stimuli, collection of multiple readings is necessary to estimate an individual's true state. It is important to note that ascertaining whether these critical biometric parameters are high or low is of equal and critical importance. High levels lead to aforementioned complications; low levels lead to illness, and even, death. For reasons stated above, these readings should be taken outside of the office or clinic setting and this typically means ‘out-of-office’ (typically home) measurement. For blood pressure, according to contemporary clinical practice guidelines, the mean of multiple home readings taken in the morning and evening over multiple days (typically 3-7 days) should be used for clinical decision making. Similarly, for blood glucose, multiple readings are required, typically taken pre-meal and/or two hours post-meal. Occasionally, 3 am glucose measurements are also performed.

A blood pressure or blood glucose “series” is a plurality of measurements, preferably taken multiple times a day, over a period of multiple days. In the case of home measurements, the individual performing self-measurement may lack the knowledge and/or skills to calculate the mean of a blood pressure or blood glucose series correctly. In the case of blood glucose, the complexity may be compounded because it is often necessary to perform this averaging over days to weeks, calculating these means separately for different times of day. This is because diabetes medication, including insulin, may be dosed multiple times per day. Accordingly, to impact a high or low reading at a specific time of day, a specific dose modification at a certain time of day may be required. This individual may bring a series of readings to his or her health care provider, but the provider, for lack of time, lack of knowledge, lack of interest, inconvenience or other reasons, may not calculate a mean. This represents a lost opportunity to use available data to its best advantage, and is a recognized and common cause of lack of adherence to clinical practice guidelines in the fields of hypertension and diabetes.

In the case of blood pressure and/or blood glucose tracking, there is a need for flexibility in summarizing measurements. For example, if an individual who is habitually tracking blood pressure or blood glucose wishes to determine the effects of exercise, he or she may take several readings prior to, and following, completion of a bout of exercise. In this case, one would calculate the mean of the measurements taken prior to exercise to compare to the mean derived from measurements performed after exercise. In some cases, the individual may wish to perform such monitoring over days, weeks, or months. In the clinical setting, when initiating or titrating drug treatments, serial home blood pressure or blood glucose series are performed and compared. Often, a practitioner needs to select readings from the days or weeks prior to drug initiation/titration to compare to readings taken four to six weeks later in order to assess the peak effect of a given antihypertensive or antidiabetic agent. A user may also wish to assess longer term temporal blood pressure or blood glucose trends, and may require summary data that covers months or even years.

Portable electronic devices, including those with touch sensitive displays, such as smartphones, watches, tablets, and sensors are commonly and increasingly being used by patients and providers to for clinical care delivery and by consumers to monitor health parameters. These devices can be used to electronically store, summarize, manipulate, and display graphically different biometrics, including blood pressure and/or blood glucose. They can also synchronize with cloud-based data platforms to recall blood pressure and/or blood glucose measurements and perform these summarization processes.

One limitation of current electronic blood pressure or blood glucose tracking systems is that the data are not summarized in a manner that is flexible, efficient, and concordant with best practice, as outlined in clinical practice guidelines. This leads to improper or incomplete use of blood pressure or blood glucose data. In the clinical realm, the most useful blood pressure or blood glucose metric for clinical decision making is the mean value. Contemporary clinical practice guidelines across the world recommend that the mean blood pressure value be used for the purposes of making a diagnosis of high or low blood pressure, or for initiating and titrating antihypertensive treatments. Blood glucose is managed in a similar fashion, sometimes using an A1c test and sometimes using the results of multiple blood glucose readings (and sometimes both). These recommendations are based on decades of foundational research comprising observational and clinical trial data collected in millions of subjects.

Well known barriers to proper use of blood pressure or blood glucose data include instances where patients fail to record all readings, patients fail to bring in or otherwise provide the measurements to their provider, or providers fail to view the readings or fail to calculate a mean blood pressure or complete assessment of blood glucose, as recommended by contemporary guidelines. Using an electronic telemonitoring system that collects measurements that have been entered manually or via a Bluetooth-enabled blood pressure or blood glucose monitoring device addresses some of the barriers. In order to address the final barrier, calculation of the mean blood pressure or provide a full picture of circadian blood glucose measurements, it is important to ensure that the mean can be calculated flexibly from a set of blood pressure or blood glucose readings. This is because a provider may wish to know the mean blood pressure or blood glucose before or after an event, such as a medication change or a hospitalization episode. In addition, different guidelines recommend different durations for a blood pressure or blood glucose series; therefore, the method of calculating the mean must be flexible to account for these differences, which usually varies from 3-7 days for blood pressure and weeks or months for blood glucose.

Therefore, there remains a need in the art to efficiently, flexibly, and easily calculate and display a mean blood pressure or blood glucose value from a set of recordings performed by an individual and stored electronically to fully capitalize on all available data. In addition, there is a need to compare different blood pressure or blood glucose series; therefore, the process of acquiring data to calculate the mean of different series, including at different times of day, must allow for this eventuality.

This background information is provided solely to facilitate an understanding of the invention, which is described below.

### SUMMARY OF THE INVENTION

Consistent with the present disclosure, computer-implemented systems and methods, and computer program products are provided for manipulating a database for search queries. The database may comprise point-of-care diagnostic health data values. Embodiments consistent with the present disclosure include computer-implemented systems and methods, and computer program products allowing a user to obtain meaningful data from the database with simple gesture or pointer control techniques.

In one exemplary embodiment, a computer-implemented method is provided for summarizing one or more series of point-of-care diagnostic health data values and displaying those summaries. It will be understood that the health data values are stored in a memory in association with a calendar date on which the health data value was acquired. The method is performed by at least one processor, and comprises the steps of
(a) displaying, on a display device, a graphical representation of a calendar; and
(b) creating and displaying, on the display device, a data summary for a first series of the health data values in response to a user defining the first series by selecting a day or days on the graphical representation of the calendar.

In embodiments, the user selection process comprises selecting the day or days by means of a computer pointer device, or by a swipe gesture with a finger or stylus on a touch screen. In the latter case, the method comprises detecting the swipe gesture to define the day or days of the first series.

In embodiments, the health data values comprise data indicative of one or more of the following: blood pressure, heart rate, blood glucose level, body temperature, body weight, respiratory rate, and patient reported outcomes.

In embodiments, the method further comprises the step of creating and displaying, on the display device, a second data summary for a second series of the health data values for comparison with the first data summary. In such embodiments, the method may comprise displaying, on the display device, visually distinct identifiers for the first and second series in either or both of the graphical representation of the calendar and the displayed data summaries.

In embodiments, the health data values are stored in the memory in association with a time stamp or a category defined by at least one of the following: time-of-day, patient body position, post-meal, or pre-meal; and creating the first data summary is based on the time stamp or the category. In such embodiments, the first data summary may comprise the health data value at a specified time, or a mean of the health data values within a specified period of time, or a mean of health data values within the category.

In one exemplary embodiment, a system is provided for summarizing one or more series of the health data values, and displaying those summaries. The system comprises at least one memory device that stores a set of instructions, and at least one processor in communication with the at least one memory device for executing the instructions to implement one or more embodiments of the above-described method.

In another exemplary embodiment, a computer program product is provided, wherein the computer program product comprises at least one non-transitory computer readable medium storing instructions executable by at least one processor to implement one or more embodiments of the above-described method.

For example, the present invention may comprise an application on a hand-held computing device with a touch screen (e.g., a tablet computer, a smart phone, or wearable computer), which device either has a memory that stores or can access a remote memory that stores time-stamped blood pressure or blood glucose measurements, and has a display device that displays a calendar view. Preferably, those days which have assigned blood pressure or blood glucose data are highlighted in some fashion. A user may then touch a portion of the graphical display of the calendar that corresponds to one end of a desired series of blood pressure or blood glucose readings, and then move to a portion of the calendar which corresponds to the other end, using what is commonly known as a swipe gesture to define the day or days of the series.

In response, the application will then display summary statistics from the defined series. The summary statistics may include daytime mean and nighttime mean blood pressure and/or heart rate or blood glucose, and/or overall mean blood pressure and/or heart rate or blood glucose, or other diagnostically relevant information which can be produced from the series.

A user may produce summary statistics from a second series which may be displayed together with the first series summary for comparison. The summary statistics may be displayed in a numerical, tabular, or graphical summary of one or more series in order to facilitate comparisons between series, summarize circadian variations in biometric parameters, and enable guideline concordant decision making and adjustment of therapies that may specifically impact certain times of the day.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIGS. 1A-1C are block diagrams showing the relationship between data acquisition, the application (running locally, remotely, or both) on a computing device, and remote network-based data storage, according to some embodiments of the invention. FIG. 1A shows local data acquisition and storage using a patient computing device and application. FIG. 1B shows local data acquisition using a patient computing device and application, and transmission to a remote server and application. FIG. 1C shows local data acquisition without a patient computing device and application, and transmission to a remote server and application.

FIGS. 2A-2C are flow diagrams showing the data acquisition process by a user interacting with a compatible data acquisition device (FIG. 2A), an incompatible data acquisition device (FIG. 2B), and without interacting with a data acquisition device (FIG. 2C), for some embodiments of the invention.

FIGS. 2D-2G show a succession of the patient graphical user interfaces for display on a computing device, when acquiring blood pressure readings from a compatible Bluetooth-enabled blood pressure monitor, when ready to acquire a reading (FIG. 2D), after a first reading (FIG. 2E), after a one minute waiting period (FIG. 2F), and after a second reading (FIG. 2G), according to some embodiments of the invention. FIG. 2H shows the graphical user interface when manually entering a blood pressure reading, according to some embodiments of the invention.

FIGS. 3A-3D illustrate the calendar GUI display of the application reviewing retrieved blood pressure data, during interaction with the calendar to create a series, and comparing two series, according to some embodiments of the invention. The figures show the calendar GUI display before a series is created (FIG. 3A), after a first series is created (FIG. 3B), after displaying more detailed information from the first series (FIG. 3C), and after a second series is created (FIG. 3D).

FIGS. 3E-3G illustrate the calendar GUI display of the application reviewing retrieved blood glucose data, during interaction with the calendar to create a series, and comparing two series, according to some embodiments of the invention. The figures show the calendar GUI display after a first series is created (FIG. 3E), after displaying more detailed information from the first series (FIG. 3F), and after a second series is created (FIG. 3G).

FIG. 4 is a flow diagram that illustrates the series creation process for health data.

FIG. 5 is a schematic depiction of one embodiment of a system of the present invention.

### DETAILED DESCRIPTION

Before explaining certain embodiments of the present disclosure in detail, it is to be understood that the disclosure is not limited in its application to the details of construction and to the arrangements of the components set forth in the following description or illustrated in the drawings. The disclosure is capable of embodiments in addition to those described and of being practiced and carried out in various ways. Also, it is to be understood that the phraseology and terminology employed herein, as well as in the abstract, are for the purpose of description and should not be regarded as limiting.

As such, those skilled in the art will appreciate that the conception and features upon which this disclosure is based may readily be utilized as a basis for designing other structures, methods, and systems for carrying out the several purposes of the present disclosure. Furthermore, the claims should be regarded as including such equivalent constructions insofar as they do not depart from the spirit and scope of the present disclosure.

### Definitions

All terms not specifically defined herein have their common, art-accepted meanings, in the context of health data monitoring, and in particular, blood pressure or blood glucose measurement and monitoring. As used herein, the following terms have the following meanings.

“Computing device”, “computer”, “processor” and like terms refer to one or more electronic devices capable of performing operations on data. Non-limiting examples of computer devices include devices referred commonly referred to as processors, servers, general purpose computers, personal computers, desktop computers, laptop computers, handheld computers, smart phones, tablet computers, and the like. Any kind of computer device adapted for carrying out the methods described herein may be used.

“Display device”, “display” and like terms in relation to a computer device, refers to any electronic device capable of presenting information in visual form. Non-limiting examples of display devices include electronic monitors and display panels regardless of their underlying technology (e.g., CRT, LED, LCD, PDP, and the like). A “graphical user interface” or “GUI” means a visual interface displayed by a display device which allows for a user to interact with an electronic computing device using icons, menus and other visual indicator (graphics) representations to display information and related user controls, unlike text-based interfaces, where data and commands are in text. GUI representations are typically manipulated by a pointing device such as a mouse, trackball, stylus, or a finger on a touch screen.

“Computer readable medium”, “CRM”, “memory”, “storage” and like terms refer to a non-transitory, tangible medium capable of persistently encoding information in a format readable by a computing device. Non-limiting examples of CRMs and memory include magnetic media (e.g., a magnetic diskette or tape), optical media (e.g., an optical disc), and solid-state media using integrated circuits (e.g., flash memory). “Computer program product” refers to a computer-readable medium storing a set of instructions in any language, code or notation, that causes a computer device to perform a particular function, whether directly or indirect after conversion to another language, code or notation.

“Communication network” refers to a network enabling electronic communications between computer devices. Non-limiting examples of communication networks include one or a combination of the Internet, a local area network or organization intranet, and a telephone network, whether wired or wireless.

The use of the terms “computer device”, “computer readable medium”, “computer program product” and “communication network” in the singular, and depiction of such elements as a single physically discrete element include the possibility of such computer elements comprising multiple physically discrete devices operatively connected to each other. Accordingly, the present invention may be implemented in a centralized fashion by a single physically discrete computer element, or in a distributed fashion by multiple physically discrete computer elements, which are operatively connected to each other, and which may be remotely situated from each other.

### Computing Devices and Health Data Acquisition Devices

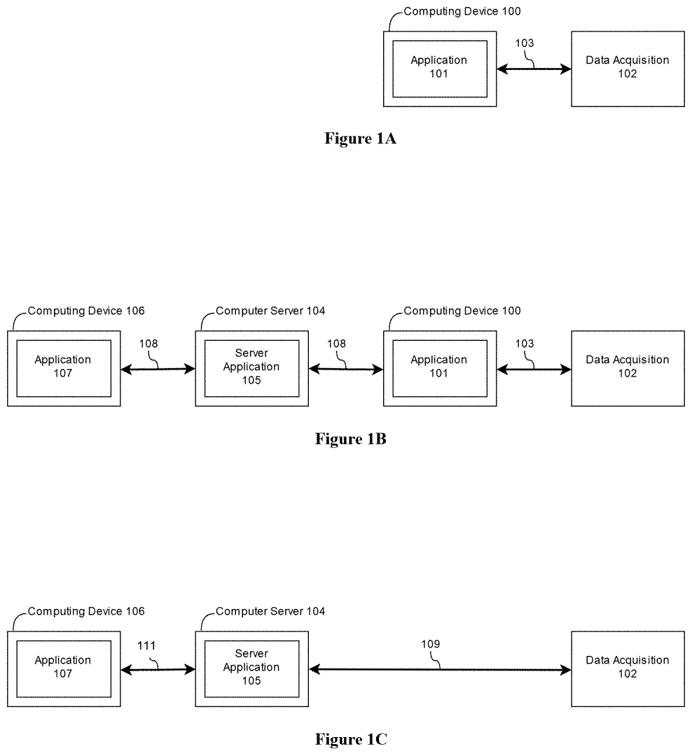

FIGS. 1A through 1C show the relationship between an application 101, 107 and a data acquisition device or system 102 for the acquisition of physiological data (health data). As used herein, “health data” may be any medically or physiologically relevant data which may be acquired by a device based on a measurements of a patient, including data such as blood pressure, heart rate, blood glucose level, body temperature, body weight, oxygen saturation, spirometry, respiratory rate, and/or patient-reported outcome measures (PROMs) such as pain level, health-related quality of life.

In FIGS. 1A and 1B, a patient acquires the health data using a data acquisition device 102, and transfers 103 the data to an application 101 operating on a patient computing device 100, which is used by the patient. The application 101 stores the received health data in the persistent storage of the device 100 for later retrieval and analysis. Alternatively, the application 101 may transmit the data to a remote location for storage. The health data is stored in association with a calendar date on which the health data is acquired.

The patient computing device 100 is a computing device with human interface devices for input and output, including general-purpose computing devices such as a cellular smart phone, tablet computer, laptop computer, wearable computer, or desktop computer. The patient computing device 100 may also have capabilities that allow it to communicate directly with compatible data acquisition devices 102 and/or with a remote computer server 104 (FIG. 1B), using any known networking protocol. The application 101 may be a standalone executable application, a web-based application, a mobile application such as an Android™ or iOS™ based application, or any other application that can be executed on patient computing device 100, directly or in conjunction with another application, such as a web browser.

The data acquisition device 102 can be any suitable device, such as an oscillometric blood pressure monitor, a blood glucose monitor, digital thermometer, or other device which reports data in digital format. In some embodiments, the patient may use an analogue tool, such as a mercury thermometer, or may report a value that is self-reported by the patient, such as a rating on the NRS-11 pain scale, in which case the data may be manually entered into the application 101 by the patient.

For compatible electronic data acquisition devices 102 used for data acquisition, the transmission 103 of the acquired data to the application 101 may be performed by wired or wireless communication, including but not limited to, Ethernet, USB or other serial connection, Wi-Fi, Bluetooth, ANT, SMS, or any other suitable current or yet to be developed protocol. For incompatible electronic devices, electronic devices without integrated communication capabilities, analogue tools, and self-reported values, transmission 103 of acquired data may be accomplished by manual entry of the value displayed by the device, tool, or their own self-reported value, into the application 101 using the input capabilities of the patient computing device 100.

As shown in FIG. 1B, the application 101 transmits 108 the data to a server application 105 on a remote computer server 104. The data transmission 108 may utilize local area networks, wireless local area networks, cellular telephone networks, intranets, and/or the Internet.

Alternatively, as shown in FIG. 1C, the data acquisition device 102 transmits 109 data directly to a remote server application 105 without the intermediate presence of a local application 101 on the patient computing device 100. If the data acquisition device 102 and server application 105 cannot use compatible communication protocols or methods, the data transmission 109 may include one or more exchange devices that provide a conversion between communication protocols or methods.

In some embodiments, as is schematically illustrated in FIG. 1C, a patient computing device 100 and application 101 may not be required. For example, a data acquisition device 102 may have an integrated cellular modem or WiFi connection that allows it to upload directly to the remote computer server 104. However, a device may be present to facilitate communication between the data acquisition device and server. For example, a cellular hotspot might be used to allow a Wi-Fi enabled sensor to upload data to a server via a cellular Internet connection. Or, a device such as a home health hub might relay data received via Bluetooth from a measurement device over the Internet. In these examples, the patient does not interact with this device directly and its only function is to seamlessly and automatically relay the data from one transmission system to another.

In either scenario, a client user retrieves 111 data from the server application 105 over a network, and views/analyzes the data using application 107 on client computing device 106. Client computing device 106 may be a general-purpose computing device with human interface devices for input and output, such as a cellular phone, smart phone, tablet, laptop computer, wearable computer, or desktop computer. It must be able to communicate with a remote computer server 104, but unlike patient computing device 100, it need not communicate with data acquisition device(s) 102. Application 107 may be a standalone executable application, a web-based application, an Android™ or iOS™ based mobile application, or any other application that can be executed on client computing device 106, directly or in conjunction with another application, such as a web browser. It is possible that differences may exist between application 101 and application 107, such as, for example, application 101 having a greater focus on data acquisition, and application 107 having a greater focus on analysis and presentation.

### Data Acquisition Process

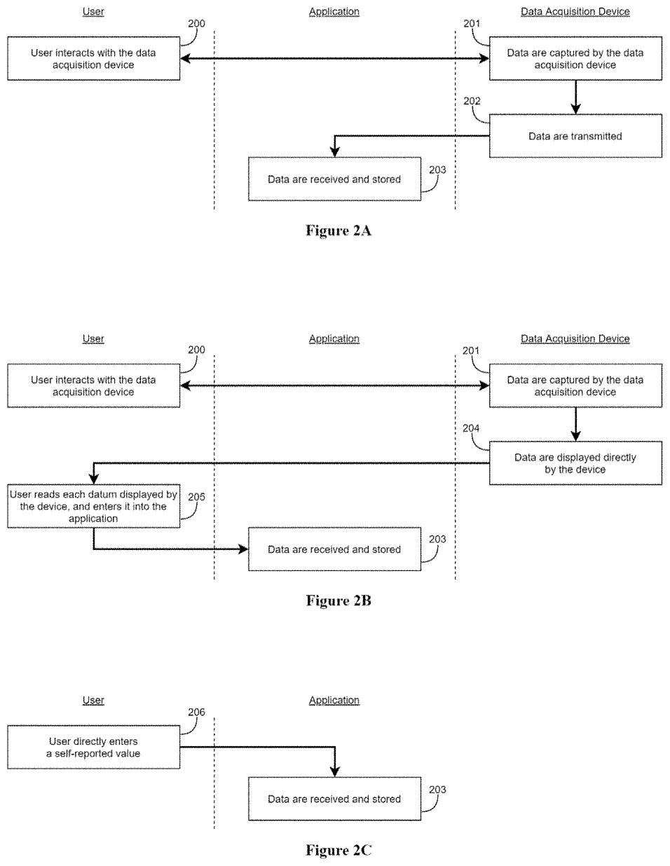

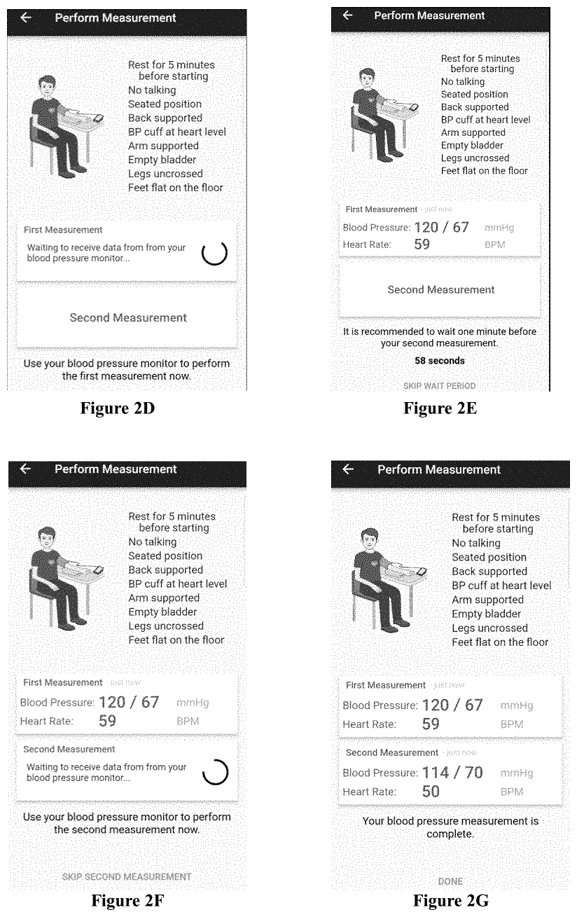

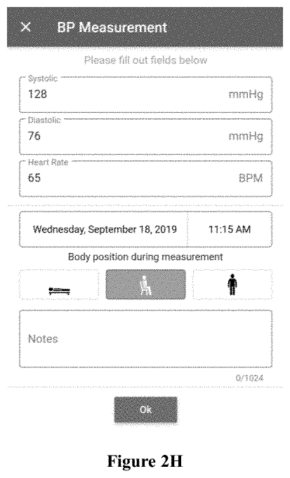

FIGS. 2A through 2C are flow diagrams which illustrate some example of a data acquisition process.

In FIGS. 2A and 2B, an interaction (step 200) occurs between the user and the data acquisition device 102 to capture data (step 201). The interaction may be initiated by the user, or the data acquisition device 102 may capture data in a continuous or passive manner. Data capture (step 201) may include acquisition of multiple data points before proceeding to the next step in the process.

In FIG. 2A, the data acquisition device 102 is a compatible electronic device with communication capabilities that allow it to transmit the data to the application (step 202). The application, which may be running on a local computing device or remotely on a computer server, receives and stores the data (step 203) for later retrieval and analysis.

Alternatively, in FIG. 2B, the data acquisition device 102 is an incompatible electronic device, an electronic device without integrated communication capabilities, or an analogue tool. In this embodiment, the data acquisition device 102 displays each datum (step 204) and the user enters the value into the application running on a local computing device (e.g., patient computing device (100)) using the device's input capabilities (step 205). The application stores the data (step 203) for later retrieval and analysis. For example, the user may obtain a blood pressure reading from a manual sphygmomanometer, a blood glucose reading from a glucometer or subcutaneous sensor, or in some other manner.

In FIG. 2C, the user does not interact with a data acquisition device (102). Here the user determines the data using a conventional method or device and directly enters a self-reported value into the application running on a local computing device (e.g., a patient computing device (100)) using the device's input capabilities (step 206). The application stores the data for later retrieval and analysis (step 203).

FIGS. 2D to 2G show successive screenshots of a graphical user interface (GUI) of an application running on a patient computing device (100), communicating with a compatible electronic data acquisition device (102), to obtain two blood pressure measurements. FIG. 2D shows the mobile app interface presented to the patient when acquiring blood pressure readings from a compatible Bluetooth-enabled blood pressure monitor serving as the data acquisition device (102). A reference image and list of instructions are shown to the patient to aid them in performing the measurement correctly. FIG. 2E shows the interface after the blood pressure reading is received from the monitor. The patient is then instructed to wait one minute before performing a second readings (they are shown a countdown). FIG. 2F shows the interface after the one minute period has completed. At this point the patient is instructed to perform the second measurement. FIG. 2G shows the interface after the second blood pressure reading is received. The patient may review the data, and then complete the process. In an analogous fashion, the same process can be repeated using a glucometer instead of a blood pressure monitor to measure blood glucose instead of blood pressure.

FIG. 2H shows the interface for manually entering the blood pressure reading, heart rate, date and time, body position, and any additional notes. Analogous manual entry of blood glucose or other biometrics can be performed.

In the examples of FIGS. 2A to 2H, the acquired health data is stored in a memory, in association with a calendar date on which the health data was acquired. In some embodiments, the health data may also be stored in association with a time stamp, or a category that indicates whether the health data was acquired at daytime, nighttime, pre-meal, post-meal, or when the patient was in a particular body position (e.g., prone, sitting or standing). This associated information may be automatically generated and assigned (e.g., using a computer calendar, and clock as shown in FIG. 2H; or based on assumed patient compliance with guidance such as shown in FIGS. 2D-2G), or manually input by the patient (e.g., see input for “Body position during measurement” as shown in FIG. 2H).

### Display of Health Data on a Calendar Interface

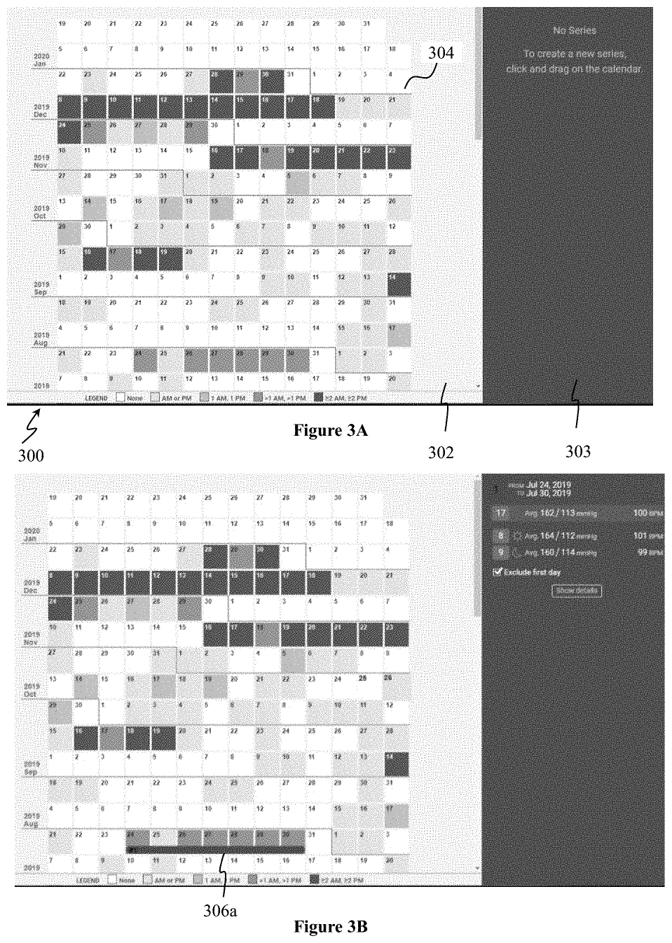

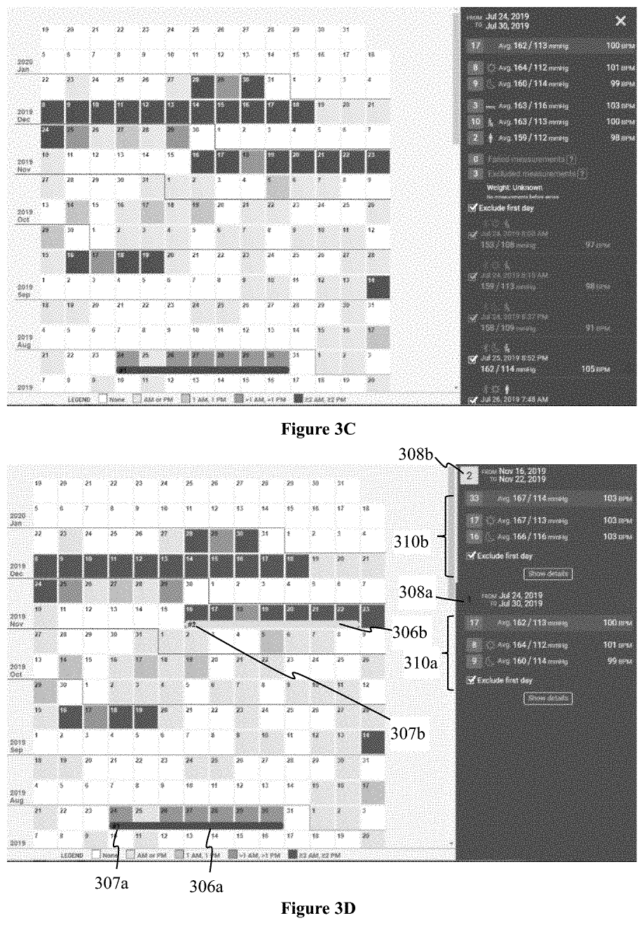

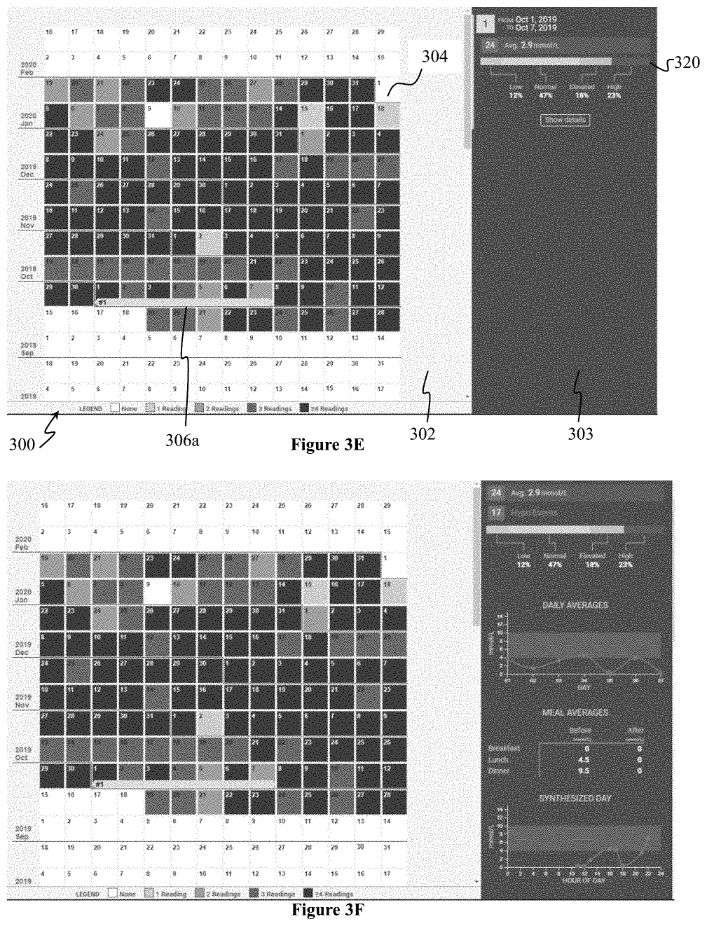

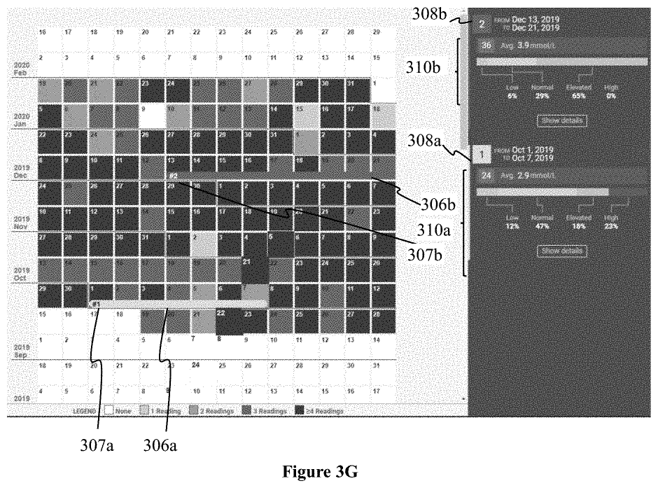

Either or both applications 101 and 107 include a graphical user interface for reviewing and analyzing data using a calendar display. FIG. 3A shows the calendar GUI of application 101 or 107 on the display 300 of device 100 or 106, respectively.

The calendar GUI of application 101 or 107 includes two sections, the calendar area 302 and the information area 303.

The calendar area 302 contains a calendar displayed as a grid of calendar squares 304, preferably where a square corresponds to a calendar day. The number of calendar squares 304 per row are not fixed and may change depending on the size of the display 300. Successive calendar squares 304 may be ordered left-to-right or right-to-left within a row, and rows may be ordered top-down or bottom-up. Additional markings and labels may be present within the calendar area 302 to indicate month and year. If the vertical size of display 300 is such that the desired number of rows cannot be displayed, the calendar area 302 may be scrolled upwards or downwards using a scroll bar, scroll wheel, keyboard keys, or swiping gestures.

The calendar squares 304 may be labelled with the day of the month, or other date, time, or calendar related values. In some embodiments, the calendar squares 304 may include extra labels, icons, or symbols related to the data that fall within that calendar day.

The calendar squares 304 are visually coded, and preferably colour-coded. The colouring of each calendar square 304 correlates with some metric calculated based on the data that fall within that calendar day. For example, in FIG. 3A, the intensity of the shading of each calendar square 304 may indicate the number of readings taken on that day. A time-shifting function may optionally be applied to each health care datum to manipulate its effective date and time before it is assigned to a calendar day. The application 101 or 107 may provide configuration options to allow users to select different colouring modes, or other visualization schemes, to visually differentiate metrics for the calendar squares 304.

The information area 303 may be used to display information related to the current state of the calendar. The information area 303 may be shown to the top, bottom, left, or right of the calendar area 302. In some embodiments the information area 303 may be modal and remain hidden until the user performs an action in the calendar area 302 which invokes the information area, causing the information area 303 to appear beside the calendar area 302, or replace the calendar area 302 until it is dismissed by the user.

Interacting with the Calendar to Create a Series and Display Data Summaries

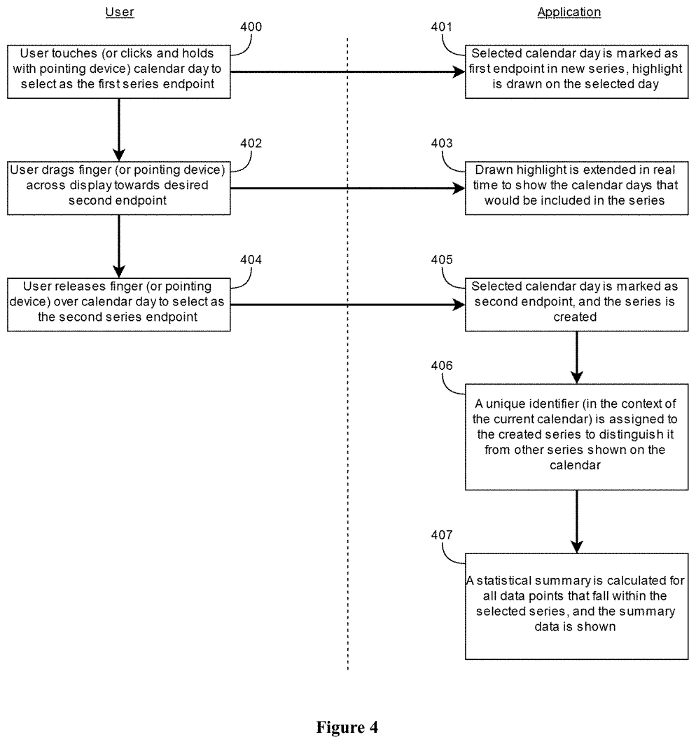

The calendar GUI of application 101 or 107 allows users to create series, which are sets of calendar days that are grouped together for analysis purposes, and which are preferably consecutive days. FIGS. 3B through 3G illustrate an exemplary interaction with the GUI, while FIG. 4 shows a schematic flowchart of the process of selecting a series by a “swipe” action on a touchscreen (e.g., using a finger, or a stylus).

FIG. 3A shows the calendar interface for a patient where data has been associated with calendar days, but no blood pressure or blood glucose series has been created. In FIG. 3B or 3E, a user has created a series by using a finger or stylus to select the end points of the series, where the device has a touchscreen; see steps 400, 402, and 404 in FIG. 4. In FIG. 3B, a visual indicator (series highlight) 306a is rendered by the application to show the calendar days that make up the series; see steps 401, 403, and 405 in FIG. 4. The calendar days that define the series can be used to retrieve relevant stored health data based on their stored association with a calendar date on which the health data was acquired.

The basic summary information may include a graphical depiction of a data summary. For example, as is shown in FIG. 3E, the proportion of “Low”, “Normal”, “Elevated” and “High” blood glucose readings, within the series denoted by series highlight 306a, are shown as colour coded segments of a bar 320.

Once a series is created, basic summary information may automatically appear, or be summoned by the user, and shown in the information area 303; see step 407 in FIG. 4. The user then has the option of selecting more detailed information from the series, as may be seen in FIG. 3C or 3F. The information can be presented in graphical, numerical, or tabular format, as shown in FIG. 3F. The more detailed information may include the total number of readings in the series, together with breakdowns into categories, such as the number of daytime and nighttime readings, pre-meal or post-meal, prone, sitting or standing readings. It is sometimes important to know blood glucose levels at a specific time, for example, 3:00 am. Category averages may also be displayed. Other health data may also be displayed, such as heart rate which is shown as BPM. A listing of each measurement along with date/time and category may be shown. Optionally, an “exclude first day” choice may be provided as some protocols recommend.

In some embodiments, “synthesized day” data may be displayed, which takes readings from all of the days in the series and averages them into one day, hour by hour, or some other specified period of time. For example, if the series comprised readings on Day 1 at 8:02 am, Day 2 at 8:29 am, and Day 5 at 8:13 am, all of those values would be averaged and shown as an “8 am” data point.

The series creation process may then be repeated to create a second series, as is shown in FIGS. 3D and 3G. The second series may have a different coloured visual indicator (series highlight) 306b than the series highlight 306a for the first series, and is assigned a unique identifier 307b (e.g., the text label “#2”) which is now displayed to distinguish it from the unique identifier 307a (e.g., the text label “#1”) of the other series; see step 406 in FIG. 4. The identifier is only unique within the context of this calendar. The identifier may be a colour, letter, character, number, icon, or other symbol. Information 310a or 310b about the series highlight 306a or 306b, respectively, including calculated summary statistics, is shown in the information area 303. The series highlight information header 308a or 308b matches the unique identifier 307a or 307b, respectively, allowing the user to correlate the series highlight information 310a or 310b with series highlight 306a or 306b, respectively, shown in the calendar area 302.

When multiple series exist in the calendar area 302, the information area 303 shows summary statistics for each series, allowing the user to compare them, as shown in FIG. 3D and FIG. 3G.

Each series highlight 306a or 306b shown in the calendar area 302 should have a corresponding series summary 310a or 310b, respectively, shown in the information area 303. Each series summary 310a or 310b has a header label 308a or 308b, respectively, which matches the identifier 307a or 307b, respectively, allowing users to determine that summary 310a or 310b corresponds to highlight 306a or 306b, respectively.

### Computer Implementation

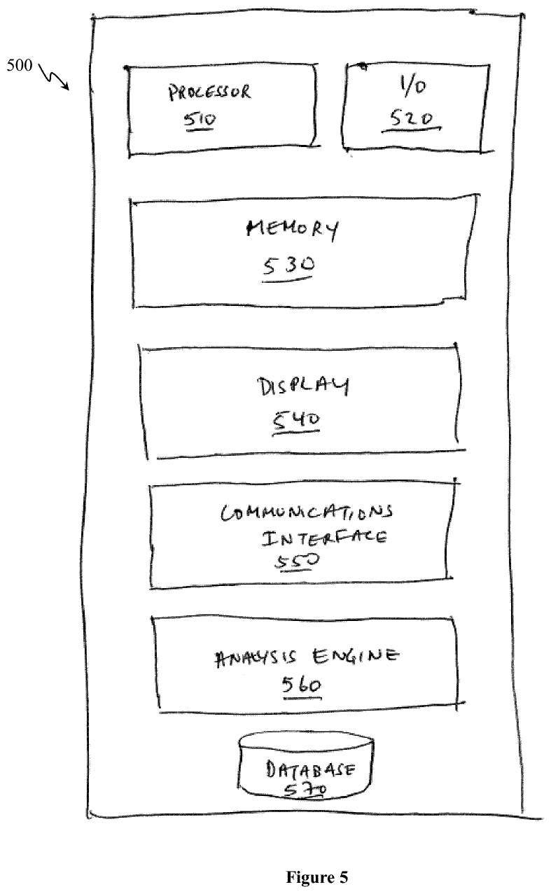

As shown in FIG. 5, system 500 may be representative of the computing device 100, computer server 104, or computing device 106, or any combination of two or more of them. The system 500 may include one or more processors 510 for executing instructions. Processors suitable for the execution of instructions include, by way of example, both general and special purpose microprocessors, and any one or more processors of any kind of digital computer. System 500 may also include one or more input/output (I/O) devices 520, which may include physical keyboards, virtual touch-screen keyboards, mice, joysticks, styluses, etc. Moreover, I/O devices 520 may include loudspeakers, handset speakers, microphones, cameras, or sensors such as accelerometers, temperature sensors, or photo/light sensors.

As further illustrated in FIG. 5, system 500 may include one or more storage devices configured to store data and/or software instructions used by the one or more processors 510 to perform operations consistent with disclosed aspects and embodiments herein. For example, system 500 may include a memory 530 configured to store one or more software programs that is executed by the one or more processors 510 to perform functions or operations, including functions or operations that enable processes (or sub-processes thereof) as described above with reference to FIGS. 2A through 4. By way of example, memory 530 may include NOR or NAND flash memory devices, Read Only Memory (ROM) devices, Random Access Memory (RAM) devices, etc. Memory 530 may also include storage mediums such as, for example, hard drives, solid state drives, tape drives, RAID arrays, etc. Although FIG. 5 shows only one memory 530, system may include any number of memories. Further, although FIG. 5 shows memory 530 as part of system 500, memory 530 may be located remotely and system 500 may be able to access memory 530 via a network.

System 500 may also include one or more display devices 540 for displaying data and information, such as the GUIs' shown in FIGS. 2A to 3G. Display 540 may be implemented using devices or technology, such as a cathode ray tube (CRT) display, a liquid crystal display (LCD), a plasma display, a light emitting diode (LED) display, a touch screen type display such as capacitive or resistive touchscreens, and/or any other type of display known in the art.

System 500 may also include one or more communications interfaces 550. Communications interface 550 may allow software and data to be transferred between computing device 100, computer server 104, and/or computing device 106 as constituent components of system 500 or otherwise, and/or other components. Examples of communications interface 550 may include a modem, a wired or wireless communications interface (e.g., an Ethernet, Wi-Fi, Bluetooth, Near Field Communication, WiMAX, WAN, LAN, etc.), a communications port (e.g., USB, IEEE 1394, DisplayPort, DVI, HDMI, VGA, Serial port, etc.), a PCMCIA slot and card, etc. Communications interface 550 may transfer software and data in the form of signals, which may be electronic, electromagnetic, optical, or other signals capable of being received by communications interface 550. These signals may be provided to communications interface 550 via a communications path (not shown), which may be implemented using wireless, wire, cable, fiber optics, radio frequency (“RF”) link, and/or other communications channels.

System 500 may include an analysis engine 560. By way of example, analysis engine 560 may be configured to summarize and manipulate data in accordance with the preceding disclosure. In some embodiments, analysis engine 560 may be implemented as at least one hardware module configured to execute the functions described herein. Alternatively, processor 510 may be configured to execute the functions of the analysis engine 560. For example, processor 510 may communicate with memory 530 that includes components of the analysis engine 560 in the form of computer-executable instructions, such that processor 510 may then execute these instructions. As another example, the functions of the analysis engine may be included in processor 510 itself such that processor 510 is configured to implement these functions.

Database 570 may be used to store data in step 203 (see FIGS. 2A-2C), and may reside on device 100, device 106, or server 104, or any combination of two or more of them.

The disclosed embodiments are not limited to separate programs or computers configured to perform dedicated tasks. For example, device 100 or 106, or server 104 may include memory 530 that stores a single program or multiple programs. Additionally or alternatively, device 100 or 106 may execute one or more programs stored on a memory 530 included with the remotely located server 104.

### Definitions and Interpretation

Aspects of the present invention may be described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The corresponding structures, materials, acts, and equivalents of all means or steps plus function elements in the claims appended to this specification are intended to include any structure, material, or act for performing the function in combination with other claimed elements as specifically claimed.

It is noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The term “another”, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described. In other words, any element or feature may be combined with any other element or feature in different embodiments, unless there is an obvious or inherent incompatibility between the two, or it is specifically excluded.

What is claimed is:

1. A system for summarizing and displaying one or more series of health data values, wherein each health data value is stored in a memory in association with a calendar date on which the health data value was acquired, the system comprising at least one memory device that stores a set of instructions, and at least one processor in communication with the at least one memory device for executing the instructions to implement a method comprising the steps of:
(a) displaying, on a display device, a graphical representation of a calendar; and
(b) creating and displaying, on the display device, a first data summary for a first series of the health data values in response to a user defining the first series by selecting a day or days on the graphical representation of the calendar.

2. The system of claim 1 wherein the display device comprises a touchscreen display, wherein selecting the day or days of the first series comprises a swipe gesture on the touchscreen display, and wherein the method comprises detecting the swipe gesture to define the day or days of the first series.

3. The system of claim 1 wherein the health data values comprise data indicative of one or more of the following: blood pressure, heart rate, blood glucose level, body temperature, body weight, respiratory rate, and patient reported outcomes.

4. The system of claim 1 wherein the method further comprises the step of creating and displaying, on the display device, a second data summary for a second series of the health data values for comparison with the first data summary.

5. The system of claim 4 wherein the method comprises displaying, on the display device, visually distinct identifiers for the first and second series in either or both of the graphical representation of the calendar and the displayed data summaries.

6. The system of claim 1 wherein the health data values are stored in the memory in association with a time stamp or a category defined by at least one of the following: time-of-day, patient body position, post-meal, or pre-meal; and wherein creating the first data summary is based on the time stamp or the category.

7. The system of claim 6 wherein the first data summary comprises the health data value at a specified time, or a mean of the health data values within a specified period of time, or a mean of health data values within the category.

8. A computer implemented method for summarizing and displaying one or more series of health data values, wherein each health data value is stored in a memory in association with a calendar date on which the health data value was acquired, the method performed by one or more processors and comprising the steps of:
(a) displaying, on a display device, a graphical representation of a calendar; and
(b) creating and displaying, on the display device, a first data summary for a first series of the health data values in response to a user defining the first series by selecting a day or days on the graphical representation of the calendar.

9. The method of claim 8 wherein the display device comprises a touch screen, wherein selecting the day or days comprises a swipe gesture on a touch screen, and the method comprises detecting the swipe gesture to define the day or days of the first series.

10. The method of claim 8 wherein the health data values comprises data indicative of one or more of the following: blood pressure, heart rate, blood glucose level, body temperature, body weight, respiratory rate, and patient reported outcomes.

11. The method of claim 8 wherein the method further comprises the step of creating and displaying, on the display device, a second data summary for a second data series of the health data values for comparison with the first data summary.

12. The method of claim 11 wherein the method comprises displaying, on the display device, visually distinct identifiers for the first and second series in either or both of the graphical representation of the calendar display and the displayed data summaries.

13. The method of claim 8 wherein the health data values are stored in the memory in association with a time-stamp or a category defined by at least one of the following: time-of-day, patient body position, post-meal or pre-meal; and wherein creating the first data summary is based on the time stamp or the category.

14. The method of claim 13 wherein the first data summary comprises the health data value at a specified time, a mean of the health data values within a specified period of time, or a mean of the health data values within the category.

15. A computer program product for summarizing and displaying one or more series of health data values, wherein each health data value is stored in a memory in association with a calendar date on which the health data value was acquire, the computer program product comprising at least one non-transitory computer-readable medium storing instructions that, when executed by at least one processor, cause the at least one processor to implement a method comprising the steps of:
(a) displaying, on a display device, a graphical representation of a calendar; and
(b) creating and displaying, on the display device, a first data summary for a first series of the health data values in response to a user defining the first series by selecting a day or days on the graphical representation of the calendar.

16. The computer program product of claim 15 wherein the display device comprises a touchscreen display, wherein selecting the day or days of the first series comprises a swipe gesture on the touchscreen display, and wherein the method comprises detecting the swipe gesture to define the day or days of the first series.

17. The computer program product of claim 15 wherein the health data values comprises data indicative of one or more of the following: blood pressure, heart rate, blood glucose level, body temperature, body weight, respiratory rate, and patient reported outcomes.

18. The computer program product of claim 15 wherein the method further comprises the step of creating and displaying, on the display device, a second data summary for a second data series of the health data values for comparison with the first data summary.

19. The computer program product of claim 18 wherein the method comprises displaying, on the display device, visually distinct identifiers for the first and second series in either or both of the graphical representation of the calendar and the displayed data summaries.

20. The computer program product of claim 15 wherein the health data values are stored in the memory in association with a time stamp or a category defined by at least one of the following: time-of-day, patient body position, post-meal, or pre-meal; and wherein creating the first data summary is based on the time stamp or the category.

21. The computer program product of claim 20 wherein the first data summary comprises the health data value at a specified time, or a mean of the health data values within a specified period of time, or a mean of health data values within the category.
